(19) United States
(12) Patent Application Publication
(10) Pub. No.: US 2023/0355581 A1
(43) Pub. Date: Nov. 9, 2023

(54) METHODS OF TREATMENT FOR MELANOMA

(71) Applicant: THE BOARD OF REGENTS OF THE UNVERSITY OF TEXAS SYSTEM, Austin, TX (US)

(72) Inventor: Murat Can COBANOGLU, Austin, TX (US)

(73) Assignee: THE BOARD OF REGENTS OF THE UNVERSITY OF TEXAS SYSTEM, Austin, TX (US)

(21) Appl. No.: 18/013,685

(22) PCT Filed: Jul. 2, 2021
(86) PCT No.: PCT/US2021/040277

§ 371 Date: Dec. 29, 2022

Related U.S. Application Data
(60) Provisional application No. 63/047,383, filed on Jul. 2, 2020.

(51) Int. Cl. A61K 31/404; A61P 35/00

(52) U.S. Cl. CPC A61K 31/404; A61P 35/00

(57) ABSTRACT

The disclosure relates to methods of treating melanoma with kinase inhibitors. Methods of treating melanoma harboring wild-type or mutant BRAF with SU11652 are provided.

### PRIORITY

This application claims the benefit of U.S. Pat. No. 63/047,383, filed on Jul. 2, 2020, which is incorporated by reference in its entirety herein.

### TECHNICAL FIELD

The present disclosure relates generally to treatment of melanoma.

### BACKGROUND

Melanoma, also known as malignant melanoma, is the most dangerous form of skin cancer. Melanoma develops from melanocytes, cells that produce the pigment melanin. Melanoma can spread more rapidly to organs than other types of skin cancer if not treated at an early stage. Risk factors for melanoma include fair skin, a history of sunburn, excessive exposure to ultraviolet (UV) light, having many moles or unusual moles, a family history of melanoma, and a weakened immune system.

More than half of melanomas harbor a mutation in the B-Raf (BRAF) gene. Combination therapies of BRAF and MEK inhibitors can be administered to improve progression-free survival. However, resistance to BRAF and MEK inhibitors develops frequently, and there are currently no targeted therapies available for BRAF-WT melanoma. Thus, there exists a need for melanoma therapies.

### SUMMARY

The present disclosure relates to the discovery of SU11652 as a therapeutic for melanoma.

Provided herein are methods of treating melanoma comprising administering to a subject having melanoma an effective amount of SU11652 (5-[(Z)-(5-chloro-2-oxo-1,2-dihydro-3H-indol-3-ylidene)methyl]-N-[2-(diethylamino)ethyl]-2,4-dimethyl-1H-pyrrole-3-carboxamide). Cells of the melanoma can comprise wild-type BRAF or mutant BRAF. Mutant BRAF can comprise, for example, a V600E mutation, a V600K mutation, a V600R mutation, a V600D mutation, a V600M mutation, a V600G mutation, a D594N mutation, a D594G mutation, a D594V mutation, a D594E mutation, a L597Q mutation, a L597R mutation, a L597S mutation, a K601E mutation, any other BRAF mutation, or a combination thereof. In an embodiment, administering SU11652 inhibits, for example, MAP3K11 activity, PAK1 activity, VEGFR2 (i.e., KDR) activity or combinations thereof. Administering SU11652 can be cytotoxic to melanoma cells. Cytotoxicity of SU11652 can be specific for melanoma cells. In an embodiment, administering SU11652 is not cytotoxic to non-cancer cells and is non-cytotoxic to breast cancer cells. The non-cancer cells can be, for example, fibroblasts, stromal cells, immune cells, or combinations thereof. The immune cells can be splenocytes. The splenocytes can comprise, for example, T cells. T cell proliferation and cytokine production can be unaltered in the presence of SU11652.

Another embodiment provides methods of treating a B-Raf inhibitor and MEK inhibitor resistant melanoma comprising administering to a patient in need thereof a therapeutically effective amount of SU11652 (5-[(Z)-(5-chloro-2-oxo-1,2-dihydro-3H-indol-3-ylidene)methyl]-N-[2-(diethylamino)ethyl]-2,4-dimethyl-1H-pyrrole-3-carboxamide). The B-Raf inhibitor is dabrafenib and the MEK inhibitor is trametinib. Cells of the melanoma can comprise mutant BRAF, for example, a V600E mutation, a V600K mutation, a V600R mutation, a V600D mutation, a V600M mutation, a V600G mutation, a D594N mutation, a D594G mutation, a D594V mutation, a D594E mutation, a L597Q mutation, a L597R mutation, a L597S mutation, a K601E mutation, any other BRAF mutation, or a combination thereof.

Another embodiment provides methods of inhibiting the growth of or killing melanoma cells comprising contacting the melanoma cells with an amount of SU11652 effective to inhibit the growth of or kill the melanoma cells. Melanoma cells can comprise wild-type BRAF or mutant BRAF. Mutant BRAF can comprise, for example, a V600E mutation, a V600K mutation, a V600R mutation, a V600D mutation, a V600M mutation, a V600G mutation, a D594N mutation, a D594G mutation, a D594V mutation, a D594E mutation, a L597Q mutation, a L597R mutation, a L597S mutation, a K601E mutation, any other BRAF mutation, or a combination thereof. Contacting the melanoma cells with SU11652 can inhibit, for example, MAP3K11 activity, PAK1 activity, VEGFR2 activity, or combinations thereof. In an embodiment, contacting the melanoma cells with SU11652 is cytotoxic to the melanoma cells. Cytotoxicity of SU11652 can be specific to melanoma cells.

### BRIEF DESCRIPTION OF THE DRAWINGS

For the dose response curves below, x-axis is log(dose) of drug in μM. Y-axis is the viability ratio where 1=100% viability, and 0=0% viability. The lines are non-linear fits, which are used to show the best fit curves. Those same fits are also used to calculate IC50 values. RMIC (where applicable) and DMSO are indicated as separate dots.

FIG. 1 panels A-F show the effect of SU11652 on BRAF V600E mutant (A375, M481) and BRAF-WT (M405, M498) as well as B16BL6 and B16F10 melanoma cells. The indicated cell line was cultured in collagen matrix to create physiologically relevant 3D culture conditions. The drug treatment was applied for 72 h and the response quantified. Three types of signal were measured using three different markers: apoptosis (CellEvent Caspase 3/7), permeabilized cell nuclei (ethidium homodimer), and all nuclei (Hoechst 33342) using an epifliorescence microscope. Each well of a 96-well plate was imaged at three different locations, with a z-step size of 2 μm for 251 steps to cover the entire height at that location. The images were processed using our software FoRTE (github.com/Cobanoglu-Lab/FoRTE) as described in Murali et al., BMC Cancer, 2019 to acquire absolute pixel counts from each channel, as well as intersections between the channels. To resolve overlaps, we used the following precedence: permeabilization indicates cell death thus overwrites both other channels; in the absence of permeabilization, apoptosis marker overrides the Hoechst nuclear stain. These absolute pixel counts signal were converted to a relative viability signal by calculating the ratio of ethidium homodimer plus apoptosis signal to the total nuclear stain signal. This viability ratio was plotted as a function of the logarithm of the dose of the drug administered (in μM units). Non-linear curves were fitted and used to calculate half maximal inhibitory concentration (IC50) values reported in the figure. In each figure, the dot shows the mean ratio for that dose while the error bars show the standard error of the mean estimate. SU11652 produced a strong cytotoxic effect on all four cell lines tested, including the two BRAF WT cell lines.

FIG. 2 panel A shows the effect of SU11652 on human foreskin fibroblast cells (HFF1), which are normal stromal cells that normally reside in the skin tissue. The data was collected and plotted using the same methodology as FIG. 1. FIG. 2 panel B shows the effect of SU11652 on human peripheral blood mononuclear cells (PBMC) isolated from human blood. These cells were cultured in suspension in 24 well ultra-low adhesion dishes, seeded at 100,000 cells per well. After 72 h of SU11652 treatment, the cells were imaged and the images were quantified using CellProfiler V2.2. For both panels, since the highest dose administered was 10 μM, estimating IC50 above that dose is theoretical and thus unreliable so IC50>10 μM is indicated in those cases. SU11652 had an IC50 above the detection range for both normal cells from the skin, and normal immune cells in the PBMC, indicating that it is unlikely to have undesirable off-target toxic effects either for normal cells in the human skin or normal immune cells.

FIG. 2 panels C-E show the effect of SU11652 on breast cancer cell lines and breast cancer cell lines measured and plotted using the same methodology as FIG. 1. The lack of efficacy on three unrelated cancer cell lines shows that the activity of SU11652 is not through a non-specific pathway broadly shared across many different cell types.

FIG. 3 panel A shows the structures of Sunitinib and SU11652. The one atom difference between the structures is shown (circled).

FIG. 3 panel B shows estimated IC50 values for sunitinib and SU11652 for the indicated target protein. SU11652 targets PAK1 while sunitinib does not. PAK1 is a validated melanoma target, both in BRAF WT melanoma (Ong et al., J. Natl. Cancer Inst. 105, 606-607 (2013)) and BRAF mutant melanoma resistant to BRAF inhibition (Babagana et al., Mol. Carcinog. 56, 1515-1525 (2017); Lu et al., Nature 550, 133-136 (2017)). SU11652 and sunitinib show differential effects on this target in biochemical assays as indicated. SU11652 also targets MAP3K11 activity more effectively than sunitinib.

FIG. 4 panels A and B show that SU11652 does not kill normal immune cells. Murine splenocytes were stimulated with anti-CD3 and anti-CD28 in addition to the indicated SU11652 doses for 2 and 3 days. At indicated timepoints, splenocytes were counted and cell death was quantified by trypan blue staining using automated cell counter. Error bars represent standard error of means for N=3 mice per timepoint.

FIG. 5 panels A-D show that SU11652 does not impair T lymphocyte proliferation, or cytokine production capacity upon activation. Murine splenocytes were stimulated with anti-CD3, anti-CD28 and murine recombinant IL-2 in addition to the indicated SU11652 doses for 3 days. Prior to stimulation, cells were stained with carboxyfluorescein succinimidyl ester (CFSE) to track cell division. Increased concentrations of SU11652 did not impair cell division in both CD8+ and CD4+T lymphocytes. Additionally, cells were treated with phorbol 12-myristate 13-acetate (PMA)/ionomycin/monensin for six hours to detect the intracellular cytokines 72 h post-stimulation. CD8+ and CD4+ lymphocytes retained their TNFα and IFNγ production abilities. CFSE dilution and the intracellular cytokines were detected by flow cytometry. Error bars represent SEM for n=3 mice.

FIG. 6A shows reduction of tumor growth in an in vivo model of melanoma in NOD scid gamma (NSG) mice. A375 cells were subcutaneously implanted at 3 million cells per mice with matrigel in the lower back of the mice. Once the tumors were palpable (150-200 mm³), the mice were randomized and drug treatment was initiated. The mice were treated with vehicle control, 20 mg/kg sunitinib, 20 mg/kg SU11652, 50 mg/kg dabrafenib+0.5 mg/kg trametinib. The mice were treated with drugs daily for the duration of the experiment. Tumor sizes were assessed twice weekly using caliper measurement, and tumor volume was calculated using the formula (length×width²×π)/6. Mice were euthanized when the tumor volume exceeded 1250 mm³ or when necessary for animal welfare. The drug was dissolved in 0.5% w/v carboxymethylcellulose sodium, 1.8% w/v NaCl, 0.4% w/v Tween-80, 0.9% w/v benzyl alcohol. All mice were sacrificed before tumor weight exceeded 10% of body weight. SU11652 significantly controlled tumor growth, and it was more effective than sunitinib.

FIG. 6B shows that SU11652 or another treatment did not incur a major toxicity that would lead to weight loss.

FIG. 6 C shows that SU11652 had greater tumor growth delay, better average tumor growth inhibition and longer time to endpoint than the closely related sunitinib.

FIG. 7 panels A-D show the effect of SU11652 on BRAF and MEK inhibitor resistant (BRAFr/MEKr) melanoma cells. M481 and A375 cells were treated with 10 nM Dabrafenib+1 nM Trametinib (M481 and A375), or 100 nM Dabrafenib+10 nM Trametinib (M481 and A375) for approximately one month to make them resistant to the BRAF and MEK inhibitor (BRAFr/MEKr). Then, SU11652 was administered at the indicated doses, and viability was measured and plotted in the same manner as indicated in FIG. 1. SU11652 was effective on these RAF/MEK dual inhibition resistant cells, showing that the mechanism of action of SU11652 is independent of the RAF/MEK inhibitors.

FIG. 8 panels A-B show a Trametinib dose response of BRAF-WT melanoma cells. Dose response viability curves were performed for M405 (A) and M498 (B) BRAF-WT melanoma cells to determine the effect of Trametinib alone along with a DMSO negative control. The data was collected and plotted in the same manner as indicated in FIG. 1. The results show that the MEK inhibitor trametinib alone is not effective in controlling BRAF WT tumor. Mutant BRAF inhibitors are also inapplicable in this context since these are BRAF WT cells. Therefore, these results show that there are not reliable treatment alternatives for BRAF WT cells.

FIG. 9A shows the reduction of tumor growth in an in vivo model of the BRAF WT M405 melanoma tumor cell model upon treatment with SU11652 on NSG mice. Cells were subcutaneously implanted at 3 million cells per mice with matrigel in the lower back of the mice. Once the tumors were palpable (150-200 mm³), the mice were randomized and drug treatment was initiated. The mice were treated with vehicle control, 20 mg/kg sunitinib and 20 mg/kg SU11652. The mice were treated with drugs daily for the duration of the experiment. Tumor sizes were assessed twice weekly using caliper measurement, and tumor volume was calculated using the formula (length×width²×π)/6. Mice were euthanized when the tumor volume exceeded 1250 mm³ or when necessary for animal welfare. The drug was dissolved in 0.5% w/v carboxymethylcellulose sodium, 1.8% w/v NaCl, 0.4% w/v Tween-80, 0.9% w/v benzyl alcohol. All mice were sacrificed before tumor weight exceeded 10% of body weight.

FIG. 9B shows that SU11652 or another treatment arm did not incur a major toxicity that would lead to weight loss.

FIG. 9C shows that SU11652 had greater tumor growth delay, better average tumor growth inhibition and longer time to endpoint than the closely related sunitinib.

FIGS. 10A-10B show in vivo dose-dependent improvement in tumor control upon administration of SU11652 in BRAF-WT tumor implanted mice. Higher doses of SU11652 lead to more efficient tumor control. WT C57BL/6 mice were implanted with 300,000 B16-F10 cells intradermally. The animals were regrouped to normalize the tumor volume. Animals were administered vehicle control (DMSO in PBS, 0 mg/kg SU11652) or increasing doses of SU11652 (10-20-40 mg/kg) orally for 4 consecutive days starting day 7, when the tumors are palpable. The tumor dimensions were measured by caliper (in mm) and the tumor volumes are calculated as follows: tumor volume=(short side²×long side)/2 (in mm³). FIG. 10 panel B compares the mean tumor burden of animals at day 7 and 11, which shows that increased SU11652 doses decreased the mean tumor burden more effectively. Tumors grew approximately 7.92 times between day 7 and 11 in 0 mg/kg group, however the presence of SU11652 decreased the tumor growth rate to 3.8 to 2.35 (10 mg/kg to 40 mg/kg).

FIG. 10C shows the tumor burden measured at days 7, 11, 14, 17 and 20 (every three days) after tumor administration as indicated. The experiment was terminated at day 20 since some animals had tumors large tumors in 0 mg/kg group and were required to be euthanized. Increasing doses of SU11652 led to increasingly effective control of tumor volume.

FIG. 11 shows SU11652 dose response of BRAF V600E melanoma cell lines resistant to treatment with the RAF/MEK Inhibitor Combo (RMIC) in 2D. The results suggest that the effect of SU11652 under 2D conditions does not match that observed in 3D and in vivo.

FIGS. 12A-12B show the (A) cytotoxic and (B) cytostatic activity of SU11652 was tested on both RAC1^{P29S} knock-in A375 and the IGR1 (already RAC1^{P29S}) cell lines. RMIC alone did not have any cytotoxic activity, but only some cytostatic activity on these cell lines with hyperactivated RAC1. However, the addition of SU11652 caused both cytotoxic and cytostatic effect in both cell lines.

### DETAILED DESCRIPTION OF THE INVENTION

The present disclosure is based on the discovery that the Sunitinib analog SU11652 can inhibit the growth of and kill melanoma cells harboring wild-type or mutant BRAF.

SU11652

Methods of treating melanoma are provided herein. The methods can comprise administering to a subject having melanoma an effective amount of SU11652 (5-[(Z)-(5-chloro-2-oxo-1,2-dihydro-3H-indol-3-ylidene)methyl]-N-[2-(diethylamino)ethyl]-2,4-dimethyl-1H-pyrrole-3-carboxamide). An analog of Sunitinib (SU11248), SU11652 is a pyrrole-indolinine compound. SU11652 can inhibit tyrosine kinase receptors, sphingomyelin phosphodiesterase, and angiogenesis, for example.

Melanomas

Any type of melanoma can be treated with the methods provided herein. Types of melanoma include nodular melanoma, acral lentiginous melanoma, lentigo maligna, lentigo maligna melanoma, amlanotic and desmoplastic melanomas, ocular melanoma, metastatic melanoma, superficial spreading melanoma, mucosal melanoma, polypoid melanoma, melanoma with small nevus-like cells, melanoma with features of Spitz nevus, uveal melanoma, and vaginal melanoma, for example.

Melanomas treated with the methods provided herein can harbor wild-type BRAF or mutant BRAF. Melanomas harboring any BRAF mutation or combinations of BRAF mutations can be treated with the methods provided herein. Exemplary BRAF mutations include, for example, a V600E mutation, a V600K mutation, a V600R mutation, a V600D mutation, a V600M mutation, a V600G mutation, a D594N mutation, a D594G mutation, a D594V mutation, a D594E mutation, a L597Q mutation, a L597R mutation, a L597S mutation, a K601E mutation, or a combination thereof.

The methods provided herein can also be applied to treating melanoma harboring any other type of mutation. Exemplary mutations include mutations in NRAS, HRAS, KRAS, NF1, TP53, CDKN2A, PTEN, AKT1/3, PIK3CA, GNAQ, GNA11, KIT, CTNNB1, EZH2, MEK1, and others. Mutations can occur as a single mutation or in combination with any other mutation, including BRAF mutations. For example, BRAF-WT melanoma can harbor any mutation or any combination of mutations in genes other than BRAF. As another example, BRAF mutant melanoma can harbor any BRAF mutation in combination with one or more additional mutations in genes other than BRAF. Any type of mutation can be present, including point mutations, substitutions, insertions, deletions, inversions, missense mutations, nonsense mutations, frameshift mutations, translocations, chromosomal loss, or chromosomal gain, for example.

Administration of SU11652

Methods of Treatment

The methods provided herein include administering to a subject having melanoma an effective amount of SU11652. SU11652 can be administered to a subject having melanoma to treat the melanoma.

As used herein, the terms “treat,” “treatment,” “therapy,” “therapeutic,” and the like refer to obtaining a desired pharmacologic and/or physiologic effect, including, but not limited to, alleviating, delaying or slowing the progression, reducing the effects or symptoms, preventing onset, inhibiting, ameliorating the onset of a disease or disorder, obtaining a beneficial or desired result with respect to a disease, disorder, or medical condition, such as a therapeutic benefit and/or a prophylactic benefit. “Treatment,” as used herein, covers any treatment of a disease in a mammal, particularly in a human, and includes: (a) preventing the disease from occurring in a subject that can be predisposed to the disease or at risk of acquiring the disease but has not yet been diagnosed as having it; (b) inhibiting the disease, i.e., arresting its development; and (c) relieving the disease, i.e., causing regression of the disease. A therapeutic benefit includes eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. In some cases, for prophylactic benefit, compounds or compositions are administered to a subject at risk of developing a particular disease, or to a subject reporting one or more of the physiological symptoms of a disease, even though a diagnosis of the disease may not have been made. The methods of the present disclosure can be used with any mammal or other animal. In some cases, the treatment can result in a decrease or cessation of symptoms. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, the term “subject” refers to any individual or patient on which the methods disclosed herein are performed. The term “subject” can be used interchangeably with the term “individual” or “patient.” The subject can be a human, although the subject may be an animal, as will be appreciated by those in the art. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

The term “effective amount” or “therapeutically effective amount” refers to that amount of a compound or composition described herein that is sufficient to affect the intended application, including but not limited to disease treatment, as defined herein. The therapeutically effective amount can vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in a target cell. The specific dose will vary depending on the particular compound or composition chosen, the dosing regimen to be followed, whether it is administered in combination with other compounds, timing of administration, the tissue to which it is administered, and the physical delivery system in which it is carried.

Inhibition of Cellular Enzymes and Functions

Administration of SU11652 can inhibit cellular enzymes and other cellular functions. Any cellular enzyme or function can be inhibited. As an example, treatment with SU11652 can inhibit a kinase. As another example, treatment with SU11652 can inhibit lipid metabolism, such as sphingolipid metabolism, for example. As yet another example, SU11652 can inhibit angiogenesis.

Kinases can catalyze the transfer of phosphate groups to substrates such a proteins that function in signal transduction cascades, for example. Other kinase substrates include lipids, carbohydrates, amino acids, and nucleotides. In addition to cell signaling, kinases function in metabolism, protein regulation, cellular transport, secretory processes, and other cellular pathways. Mutation of kinases can result in dysregulated kinase activity and play a role in cellular transformation and cancer.

Any kinase can be inhibited, including serine-threonine kinases and tyrosine kinases, for example. Exemplary tyrosine kinases include AATK, ABL, ABL2, ALK, AXL, BLK, BMX, BTK, CSF1R, CSK, DDR1, DDR2, EGFR, EPHA1, EPHA2, EPHA3, EPHA4, EPHA5, EPHA6, EPHA7, EPHA8, EPHA10, EPHB1, EPHB2, EPHB3, EPHB4, EPHB6, ERBB2, ERBB3, ERBB4, FER, FES, FGFR1, FGFR2, FGFR3, FGFR4, FGR, FLT1, FLT3, FLT4, FRK, FYN, GSG2, HCK, IGF1R, ILK, INSR, INSRR, IRAK4, ITK, JAK1, JAK2, JAK3, KDR, KIT, KSR1, LCK, LMTK2, LMTK3, LTK, LYN, MATK, MERTK, MET, MLTK, MST1R, MUSK, NPR1, NTRK1, NTRK2, NTRK3, PDGFRA, PDGFRB, PLK4, PTK2, PTK2B, PTK6, PTK7, RET, ROR1, ROR2, ROS1, RYK, SGK493, SRC, SRMS, STYK1, SYK, TEC, TEK, TEX14, TIE1, TNK1, TNK2, TNNI3K, TXK, TYK2, TYRO3, YES1, and ZAP70. Exemplary serine-threonine kinases include AK1, ACVR1 (ALK2), ACVR1B (ALK4), ACVR2A, ACVR2B, ACVRL1 (ALK1), ADCK3, ADRBK1 (GRK2), ADRBK2 (GRK3), AKT1 (PKB ALPHA), AKT2 (PKB BETA), AKT3 (PKB GAMMA), AMPK, ANKK1, AURKA (AURORA A), AURKB (AURORA B), AURKC (AURORA C), BMPRIA (ALK3), BMPR1B (ALK6), BMPR2, BRAF, BRSK1 (SAD1), BRSK2, CAMK1, CAMK1D (CAMKI DELTA), CAMK1G (CaMKI GAMMA), CAMK2A (CAMKII ALPHA), CAMK2B (CAMKII BETA), CAMK2D (CAMKII DELTA), CAMK2G (CAMKII gamma), CAMK4 (CAMKIV), CAMKKI (CAMKKA), CAMKK2 (CAMKK BETA), CASK, CDC42BPA (MRCKB), CDC42BPB (MRCKA), CDC42BPG (MRCKG), CDC7/DBF4, CDK1/cyclin A2, CDK1/cyclin B, CDK11/cyclin C, CDK13/cyclin K, CDK14 (PFTK1)/cyclin Y, CDK16 (PCTK1)/cyclin Y, CDK17/cyclin Y, CDK18/cyclin Y, CDK2/cyclin A, CDK2/cyclin A1, CDK2/cyclin E1, CDK2/cyclin 0, CDK3/cyclin E1, CDK4/cyclin D1, CDK4/cyclin D3, CDK5/p25, CDK5/p35, CDK6/cyclin D1, CDK6/Cyclin D3, CDK7/cyclin H/MNAT1, CDK8/cyclin C, CDK9, CDK9/cyclin K, CDK9/cyclin T1, CDKL5, CHEK1 (CHK1), CHEK2 (CHK2), CHUK (IKK ALPHA), CLK1, CLK2, CLK3, CLK4, CSNKIA1 (CK1 ALPHA1), CSNK1A1L (CK1 alpha 1L), CSNK1D (CK1 DELTA), CSNK1E (CK1 EPSILON), CSNK1E (CK1 epsilon) R178C, CSNK1G1 (CK1 GAMMA 1), CSNK1G2 (CK1 GAMMA 2, CSNK1G3 (CK1 GAMMA 3), CSNK2A1 (CK2 ALPHA 1), CSNK2A2 (CK2 ALPHA 2), DAPK1, DAPK2, DAPK3 (ZIPK), DCAMKL1 (DCLK1), DCAMKL2 (DCK2), DDR1, DMPK, DNA-PK, DYRK1A, DYRK1B, DYRK2, DYRK3, DYRK4, EEF2K, EIF2AK1 (HRI), EIF2AK2 (PKR), EIF2AK3 (PERK), ERN1, ERN2, FRAP1 (MTOR), GAK, GRK1, GRK4, GRK5, GRK6, GRK7, GSG2 (HASPIN), GSK3A, GSK3B (GSK3 BETA), HIPK1 (MYAK), HIPK2, HIPK3 (YAK1), HIPK4, HUNK, ICK, IKBKB (IKK BETA), IKBKE (IKK EPSILON), IRAK1, IRAK3, IRAK4, LATS1, LATS2, LIMK1, LIMK2, LRRK2, LRRK2, KSR2, MAP2K1 (MEK1), MAP2K2 (MEK2), MAP2K3 (MEK3), MAP2K4 (MEK4), MAP2K5 (MEK5), MAP2K6 (MKK6), MAP3K10 (MLK2), MAP3K11 (MLK3), MAP3K14 (NIK), MAP3K19 (YSK4), MAP3K2 (MEKK2), MAP3K3 (MEKK3), MAP3K5 (ASK1), MAP3K7/MAP3K7IP1 (TAK1-TAB1), MAP3K8 (COT), MAP3K9 (MLK1), MAP4K1 (HPK1), MAP4K2 (GCK), MAP4K3 (GLK), MAP4K4 (HGK), MAP4K5 (KHS1), MAPK1 (ERK2), MAPK10 (JNK3), MAPK11 (P38 BETA), MAPK12 (P38 GAMMA), MAPK13 (P38 DELTA), MAPK14 (P38 ALPHA), MAPK15 (ERK7), MAPK3 (ERK1), MAPK7 (ERK5), MAPK8 (JNK1), MAPK9 (JNK2), MAPKAPK2, MAPKAPK3, MAPKAPK5 (PRAK), MARK1 (MARK), MARK2, MARK5, MARK4, MASTL, MINK1, MKNK1 (MNK1), MKNK2 (MNK2), MLCK (MLCK2), MLK4, MST4, MYLK (MLCK), MYLK2 (SKMLCK), MYLK4, MYO3A (MYO3 alpha), MYO3B (MYO3 beta), NEK1, NEK11 (FLJ23495), NEK2, NEK4, NEK6, NEK7, NEK8, NEK9, NIM1K, NLK, NUAK1 (ARKS), NUAK2, OXSR1, PAK1, PAK2 (PAK65), PAK3, PAK4, PAK6, PAK7 (KIAA1264), PASK, PBK (TOPK), PDK1, PDK4 (PDHK4), PHKG1, PHKG2, PIM1, PIM2, PIM3, PKMYT1, PKN1 (PRK1), PKN2 (PRK2), PLK1, PLK2, PLK3, PLK4, PRKACA (PKA), PRKACB (PRKAC beta), PRKACG (PRKAC gamma), PRKCA (PKC ALPHA), PRKCB1 (PKC BETA 1), PRKCB2 (PKC BETA II), PRKCD (PKC DELTA), PRKCE (PKC EPSILON), PRKCG (PKC GAMMA), PRKCH (PKC ETA), PRKCI (PKC IOTA), PRKCN (PKD3), PRKCQ (PKC THETA), PRKCZ (PKC ZETA), PRKD1 (PKC MU), PRKD2 (PKD2), PRKG1, PRKG2 (PKG2), PRKX, RIPK2, RIPK3, RIPK5, ROCK1, ROCK2, RPS6KA1 (RKS1), RPS6KA2 (RSK3), RPS6KA3 (RSK2), RPS6KA4 (MSK2), RPS6KA5 (MSK1), RPS6KA6 (RSK4), RPS6KB1 (P70S6K), RPS6KB2 (P70S6K BETA), SBK1, SGK (SGK1), SGK2, SGK3 (SKGL), SIK1, SIK3, SLK, SNF1LK2 (QIK), SRPK1, SRPK2, STK16 (PKL12), STK17A (DRAK1), STK17B (DRAK2), STK22B (TSSK2), STK22D (TSSK1), STK23 (MSSK1), STK24 (MST3), STK25 (YSK1), STK3 (MST2), STK32B (YANK2), STK32C (YANK3), STK33, STK38 (NDR), STK38L (NDR2), STK39 (STLK3), STK4 (MST1), TAOK1, TAOK2 (TAO1), TAOK3 (JIK), TBK1, TESK2 TESK1, TGFBR1 (ALK5), TGFBR2, TLK1, TLK2, TNIK, TTK, ULK1, ULK2, ULK3, VRK2, WEE1, WNK1, WNK2, WNK3, and ZAK.

In an embodiment, SU11652 inhibits MAP3K11 activity. In another embodiment, SU11652 inhibits PAK1 activity. In another embodiment SU11652 inhibits VEGFR2 activity. SU11652 can also inhibit any combination of kinases. For example, SU11652 can inhibit two or more serine-threonine kinases. As another example, SU11652 can inhibit two or more tyrosine kinases. As yet another example, SU11652 can inhibit a combination of any number of serine-threonine kinases and any number of tyrosine kinases. In an embodiment, SU11652 inhibits MAP3K11 activity, PAK1 activity, VEGFR2 activity, or combinations thereof.

In an embodiment, SU11652 inhibits sphingolipid metabolism. Enzymes that inhibit shingolipid metabolism include sphingomyelinases, for example. Exemplary shingomyelinases include any type of sphingomyelin phosphodiesterase, such as lysosomal acid sphingomyelinase, secreted acid sphingomyelinase, neutral sphingomyelinase (magnesium-dependent and magnesium-independent), and alkaline sphingomyelinase.

Cytotoxicity of SU11652

Administering SU11652 can be cytotoxic to melanoma cells. Cytotoxicity of a compound means that the compound is toxic to cells. A cytotoxic compound can kill cells that are exposed to the compound. For example, cells can undergo necrosis or apoptosis in response to treatment with a cytotoxic compound.

Cytotoxicity of a compound can be specific for a particular cell type. As used herein, specific cytotoxicity refers to a compound being cytotoxic to one particular cell type but not another. Thus, different cell types can display different sensitivities to a cytotoxic compound. Accordingly, treatment with a cytotoxic compound can result in one type of cell being killed while another type of cell is not killed in the presence of the compound. The percentage of cells killed in the presence of a cytotoxic compound can be about 10%, about 20%, about 30%, about 40%, about 50%, about 60%, about 70%, about 80%, about 90%, about 95%, about 99%, about 99.5%, about 99.9%, and any number or range in between. In an embodiment, all cells, i.e., about 100% of the cells, are killed in the presence of the cytotoxic compound. As used herein, a cell type not killed in the presence of a cytotoxic compound refers to cells remaining viable in the presence of the cytotoxic compound. The percentage of cells that remains viable can be about 40%, about 50%, about 60%, about 70%, about 80%, about 90%, about 95%, about 99%, about 99.5%, about 99.9%, and any number or range in between. In an embodiment, all cells, i.e., about 100% of the cells, remain viable.

Sensitivity to a cytotoxic compound can be different depending on cell origin, such as the organ a cell is derived from. For example, cells derived from any organ can display different sensitivity to a cytotoxic compound than cells derived from another organ. Sensitivity to a cytotoxic compound can also vary with cellular function, such as immune function, secretory function, stem and progenitor cell function, structural or support function, barrier function, and others. Cell types with different functions include epithelial cells, endothelial cells, fibroblasts, immune cells, hematopoietic cells, stromal cells, nerve cells, secretory cells, and others. In addition, cancer cells and normal or non-cancer cells can display different sensitivities to a cytotoxic compound. As an example, cytotoxicity of SU11652 can be specific for melanoma cells. Accordingly, in an embodiment, administering SU11652 is cytotoxic to melanoma cells. In another embodiment, administration of SU11652 is not cytotoxic to non-cancer cells. The non-cancer cells can be any cell type or combinations of cell types, such as fibroblasts, stromal cells, immune cells, and others. As another example of differential sensitivity, administration of SU11652 can be non-cytotoxic to certain other types of cancer cells. In an embodiment, administration of SU11652 is not cytotoxic to breast cancer cells.

Effects of SU11652 on T Cell Proliferation and Cytokine Production

Immune cells that are not killed by treatment with SU11652 can be splenocytes. Splenocytes can comprise T cells that mediate immune functions, for example. In an embodiment, immune cell proliferation is unaltered in the presence of SU11652. Immune cells that can proliferate in the presence of SU11652 include T cells, natural killer (NK) cells, B cells, dendritic cells, macrophages, monocytes, granulocytes, neutrophils, and other lymphocytes, including stem cells. T cells that can proliferate include any type of T cell, such as CD8⁺ cells, CD4⁺ cells, regulatory T cells (Tregs), memory T cells, and others. T cells can undergo any number of cell divisions in the presence of SU11652, such as 1, 2, 3, or more cell divisions in the presence of SU11652. T cells that can undergo cell divisions can be located in any organ or in any part of the body, including spleen, thymus, bone marrow, lymph nodes, and blood, for example.

In another embodiment, cytokine production is unaltered in the presence of SU11652. Cytokine production of any immune cell can be unaltered upon treatment with SU11652. Immune cells that can produce cytokines in the presence of SU11652 include T cells, natural killer (NK) cells, B cells, dendritic cells, macrophages, monocytes, granulocytes, neutrophils, and other lymphocytes, including stem cells. In an embodiment, the immune cells are T cells. T cells that can produce cytokines in the presence of SU11652 can be any type of T cell, including CD8⁺ cells, CD4⁺ cells, regulatory T cells (Tregs), memory T cells, and others. Immune cells can produce any type of cytokine in the presence of SU11652. Exemplary cytokines include interferons, TNF-α, TGF-β, G-CSF, and GM-CSF. In an embodiment, CD8⁺ cells produce TNFα, IFNγ, or both, in the presence of SU11652. In another embodiment, CD4⁺ cells produce TNFα, IFNγ, or both, in the presence of SU11652. Type I interferons such as IFN-α and IFN-β, for example, can be produced by any cell.

Immune cells and other normal or non-cancerous cells can also produce interleukins and chemokines in the presence of SU11652. Exemplary interleukins include IL-1, IL-2, IL-3, IL-4, IL-5, IL-6, IL-7, IL-8, IL-10, IL-12, IL-15, IL-18, IL-21, and IL-23; exemplary chemokines include CCL3, CCL26, and CXCL7.

Types and Doses of Administration

SU11652 can be administered by any route, including orally, intraduodenally, parenterally (including intravenous, subcutaneous, intramuscular, intravascular or by infusion), topically or rectally. SU11652 can be administered at any dose and with any frequency to produce a desired treatment outcome. For example, SU11652 can be administered in the range of about 0.001 to about 1000 mg/kg body weight/day. SU11652 can also be administered at a dose of about 1 mg, about 5 mg, about 10 mg, about 20 mg, about 30 mg, about 40 mg, about 50 mg, about 60 mg, about 70 mg, about 80 mg, about 90 mg, about 100 mg, about 125 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, and any number or range in between. Administration can be once per day, two times per day, three times per day, four times per day or more. SU11652 can be administered for one week, two weeks, three weeks, four weeks, 5 weeks, 6 weeks, 7 weeks, 8 weeks, 9 weeks, 10 weeks, 11 weeks, 12 weeks, or more. Administration for a particular period of time at a particular dose given one or more times a day can constitute a cycle of treatment. A cycle of treatment can be repeated after a recovery time of 1 week, 2 weeks, 3 weeks, 4 weeks, 5 weeks, 6 weeks, 7 weeks, 8 weeks, 9 weeks, 10 weeks, 11 weeks, 12, weeks, or more. A single cycle or more than one cycle of treatment with SU11652 can be administered. Any number of cycles can be administered for a course of treatment. A single course or more than one course of treatment with SU11652 can be administered.

Formulations

SU11652 can be formulated according to methods of administration that include, for example, oral routes, intraduodenal routes, parenteral routes (including intravenous, subcutaneous, intramuscular, intravascular or by infusion), topical administration, and rectal administration. Accordingly, SU11652 can be formulated in solid form, liquid form, or any other form suitable for the method of administration. Exemplary formulations include tablets, pills, pellets, powders, sprays, ointments, creams, capsules, solutions, syrups, drops, granules, and suppositories. In any formulation, the medicament can include a therapeutically effective amount of an active compound or composition or a pharmaceutically acceptable form thereof and a pharmaceutically acceptable carrier. As used herein, “pharmaceutically acceptable” means that which is useful in preparing a pharmaceutical composition that is generally safe, non-toxic and neither biologically nor otherwise undesirable and includes that which is acceptable for veterinary use as well as human pharmaceutical use. Types of excipients can include fillers, binders, lubricants, diluents, sweetening and flavoring agents, preservatives, disintegrators, permeation enhancers, antiadherents, coatings, coloring agents, disintegrants, glidants, preservatives, sorbents, and vehicles or carriers, for example. Suitable pharmaceutical carriers include inert diluents or fillers, water and various organic solvents, for example.

Exemplary excipients for solid formulations can include inert diluents, such as calcium carbonate, sodium carbonate, lactose, calcium phosphate or sodium phosphate, for example; granulating and disintegrating agents, such as microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. Tablets or pills can be un-coated or coated by known techniques to mask the taste of the drug or delay disintegration and absorption in the gastrointestinal tract and thereby provide a sustained action over a longer period. For example, a water soluble taste masking material such as hydroxypropyl methylcellulose or hydroxypropyl cellulose, or a time delay material such as ethyl cellulose, or cellulose acetate butyrate can be employed as appropriate. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with a water soluble carrier such as polyethyleneglycol or an oil medium, for example peanut oil, liquid paraffin, or olive oil.

Aqueous suspensions can contain the active material in admixture with excipients suitable for the manufacture of aqueous suspensions. Such excipients are suspending agents, for example sodium carboxymethylcellulose, methylcellulose, hydroxypropyl methylcellulose, sodium alginate, polyvinyl-pyrrolidone, gum tragacanth and gum acacia; dispersing or wetting agents can be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions can be formulated by suspending the active ingredient in a vegetable oil, for example arachis oil, olive oil, sesame oil or coconut oil, or in mineral oil such as liquid paraffin. The oily suspensions can contain a thickening agent, for example beeswax, hard paraffin or cetyl alcohol. Sweetening agents such as those set forth above and flavoring agents can be added to provide a palatable oral preparation. These compositions can be preserved by the addition of an anti-oxidant such as butylated hydroxyanisol or alpha-tocopherol.

Dispersible powders and granules suitable for preparation of an aqueous suspension by the addition of water can provide the active ingredient in admixture with a dispersing or wetting agent, suspending agent and one or more preservatives. Suitable dispersing or wetting agents and suspending agents are exemplified above. Additional excipients, for example sweetening, flavoring and coloring agents, can also be present. These compositions can be preserved by the addition of an anti-oxidant such as ascorbic acid.

Pharmaceutical compositions can also be in the form of an oil-in-water emulsions. The oily phase can be a vegetable oil, for example olive oil or arachis oil, or a mineral oil, for example liquid paraffin or mixtures of these. Suitable emulsifying agents can be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions can also contain sweetening agents, flavoring agents, preservatives and antioxidants.

Syrups and elixirs can be formulated with sweetening agents, for example glycerol, propylene glycol, sorbitol or sucrose. Such formulations can also contain a demulcent, a preservative, flavoring and coloring agents and antioxidant.

Pharmaceutical compositions can be in the form of a sterile injectable aqueous solution. Among the acceptable vehicles and solvents that can be employed are water, Ringer's solution and isotonic sodium chloride solution. A sterile injectable preparation can also be a sterile injectable oil-in-water microemulsion where the active ingredient is dissolved in the oily phase. For example, the active ingredient can first be dissolved in a mixture of soybean oil and lecithin. The oil solution can then be introduced into a water and glycerol mixture and processed to form a microemulsion. The injectable solutions or microemulsions can be introduced into a patient's blood-stream by local bolus injection. Alternatively, it can be advantageous to administer the solution or microemulsion in such a way as to maintain a constant circulating concentration of the instant compound. In order to maintain such a constant concentration, a continuous intravenous delivery device can be utilized. Any intravenous delivery device or intravenous pump can be used. The pharmaceutical compositions can be in the form of a sterile injectable aqueous or oleagenous suspension for intramuscular and subcutaneous administration. This suspension can be formulated according to methods known in the art using any suitable dispersing or wetting agents and suspending agents. The sterile injectable preparation can also be a sterile injectable solution or suspension in a non-toxic parenterally-acceptable diluent or solvent, for example as a solution in 1,3-butane diol. In addition, sterile, fixed oils can be employed as a solvent or suspending medium. For this purpose, any bland fixed oil can be employed, including synthetic mono- or diglycerides. In addition, fatty acids such as oleic acid find use in the preparation of injectables.

Pharmaceutical compositions can also be administered in the form of suppositories for rectal administration of the drug. These compositions can be prepared by mixing a compound, composition, or medicament such as SU11652 with a suitable non-irritating excipient that is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing a compound, composition, or medicament such as SU11652 can be used. As used herein, topical application can include mouth washes and gargles.

Pharmaceutical compositions can be administered in intranasal form via topical use of suitable intranasal vehicles and delivery devices, or via transdermal routes, using transdermal skin patches well known to those of ordinary skill in the art. Dosage administration in the form of a transdermal delivery system can be continuous rather than intermittent throughout the dosage regimen.

Methods of Treating an Inhibitor Resistant Melanoma

Provided herein, in some embodiments, are methods of treating inhibitor resistant melanoma. More than half of melanomas harbor a mutation in the B-Raf (BRAF) gene as described above. The mitogen-activated extracellular signal-regulated kinase (MEK) is part of the MAPK signaling cascade activated in melanoma and proliferation of cancerous cells. Combination therapies of BRAF and MEK inhibitors can be administered to improve progression-free survival. BRAF inhibitors are drugs that target an acquired mutation of B-RAF that is associated with cancer. BRAF inhibitors include, for example, vemurafenib (Zelboraf), dabrafenib (Tafinlar) and encorabenib (Braftovi). MEK inhibitors are drugs that deregulate MEK, block cell proliferation, and induce apoptosis. MEK inhibitors include, for example, trametinib (Mekinish), cobimetinib (Cotellic), and binimetinib (Mektovi). However, some melanomas are unresponsive to these inhibitors. Furthermore, clinical use of these inhibitors often leads to BRAF and MEK inhibitor resistance. Inhibitor resistant melanoma can be intrinsic or acquired. Intrinsic inhibitor resistant melanomas do not meet criteria for threshold response to BRAF inhibitors. Acquired inhibitor resistance melanomas show short-lived clinical responses followed by relapse and tumor progression. Currently, there are no targeted therapies for inhibitor resistant melanoma. Thus, there exists a need for inhibitor resistant melanoma treatments.

In an embodiment, a B-Raf inhibitor and MEK inhibitor resistant melanoma can be treated by administering to a patient in need thereof a therapeutically effective amount of SU11652 (5-[(Z)-(5-chloro-2-oxo-1,2-dihydro-3H-indol-3-ylidene)methyl]-N-[2-(diethylamino)ethyl]-2,4-dimethyl-1H-pyrrole-3-carboxamide). SU11652 can be administered at about the same time as the inhibitors or after treatment with inhibitors (e.g., 1, 2, 3, 4, 8, 12, 24, 48 or weeks more after inhibitor treatment).

Methods of Inhibiting Growth of or Killing Melanoma Cells

Provided herein, in some embodiments, are methods of inhibiting the growth of or killing melanoma cells. Methods of inhibiting the growth of or killing melanoma cells can comprise contacting the melanoma cells with an amount of SU11652 effective to inhibit the growth of or kill melanoma cells.

Inhibiting the growth of cells means that cell growth and cell division is inhibited. Thus, a compound or composition that inhibits cell growth and cell division can be a cytostatic compound or composition. Inhibiting cell growth can result in cells dying by apoptosis or necrosis, for example. In an embodiment, cell growth and cell division of about 10%, about 20%, about 30%, about 40%, about 50%, about 60%, about 70%, about 80%, about 90%, about 95%, about 99%, about 99.5%, about 99.9%, and any number or range in between, is inhibited. In another embodiment, there is no cell growth and no cell division, i.e., cell growth and cell division of about 100% of cells is inhibited. Inhibiting cell growth can also result in differentiation of cells. Differentiation of cells can turn a cancer cell, such as a melanoma cell and other cancer cells, into a less immature cell. In an embodiment, about 10%, about 20%, about 30%, about 40%, about 50%, about 60%, about 70%, about 80%, about 90%, about 95%, about 99%, about 99.5%, about 99.9%, and any number or range in between, of cells can differentiate in the presence of SU11652. In another embodiment, all cells or about 100% of cells can differentiate in the presence of SU11652.

The procedures described herein employ, unless otherwise indicated, conventional techniques of chemistry, molecular biology, microbiology, recombinant DNA, genetics, immunology, cell biology, cell culture and transgenic biology, which are within the skill of the art. (See, e.g., Maniatis, et al., Molecular Cloning, Cold Spring Harbor Laboratory Press, Cold Spring Harbor, N.Y. (1982); Sambrook, et al., (1989); Sambrook and Russell, Molecular Cloning, 3rd Ed., Cold Spring Harbor Laboratory Press, Cold Spring Harbor, N.Y. (2001); Ausubel, et al., Current Protocols in Molecular Biology, John Wiley & Sons (including periodic updates) (1992); Glover, DNA Cloning, IRL Press, Oxford (1985); Russell, Molecular biology of plants: a laboratory course manual, Cold Spring Harbor Laboratory Press, Cold Spring Harbor, N.Y. (1984); Anand, Techniques for the Analysis of Complex Genomes, Academic Press, N Y (1992); Guthrie and Fink, Guide to Yeast Genetics and Molecular Biology, Academic Press, N Y (1991); Harlow and Lane, Antibodies, Cold Spring Harbor Laboratory Press, Cold Spring Harbor, N.Y. (1988); Nucleic Acid Hybridization, B. D. Hames & S. J. Higgins eds. (1984); Transcription and Translation, B. D. Hames & S. J. Higgins eds. (1984); Culture of Animal Cells, R. I. Freshney, A. R. Liss, Inc. (1987); Immobilized Cells And Enzymes, IRL Press (1986); B. Perbal, A Practical Guide To Molecular Cloning (1984); the treatise, Methods In Enzymology, Academic Press, Inc., NY); Methods In Enzymology, Vols. 154 and 155, Wu, et al., eds.; Immunochemical Methods In Cell And Molecular Biology, Mayer and Walker, eds., Academic Press, London (1987); Handbook of Experimental Immunology, Volumes I-IV, D. M. Weir and C. C. Blackwell, eds. (1986); Riott, Essential Immunology, 6th Edition, Blackwell Scientific Publications, Oxford (1988); Fire et al., RNA Interference Technology From Basic Science to Drug Development, Cambridge University Press, Cambridge (2005); Schepers, RNA Interference in Practice, Wiley-VCH (2005); Engelke, RNA Interference (RNAi): The Nuts & Bolts of siRNA Technology, DNA Press (2003); Gott, RNA Interference, Editing, and Modification: Methods and Protocols (Methods in Molecular Biology), Human Press, Totowa, N.J. (2004); and Sohail, Gene Silencing by RNA Interference: Technology and Application, CRC (2004)).

The compositions and methods are more particularly described below and the Examples set forth herein are intended as illustrative only, as numerous modifications and variations therein will be apparent to those skilled in the art. As used in the description herein and throughout the claims that follow, the meaning of “a”, “an”, and “the” includes plural reference unless the context clearly dictates otherwise. The term “about” in association with a numerical value means that the value varies up or down by 5%. For example, for a value of about 100, means 95 to 105 (or any value between 95 and 105).

The terms used in the specification generally have their ordinary meanings in the art, within the context of the compositions and methods described herein, and in the specific context where each term is used. Some terms have been more specifically defined below to provide additional guidance to the practitioner regarding the description of the compositions and methods.

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising”, “consisting essentially of”, and “consisting of” may be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

Any single term, single element, single phrase, group of terms, group of phrases, or group of elements described herein can each be specifically excluded from the claims.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the invention are described in terms of Markush groups or other grouping of alternatives, those skilled in the art will recognize that the invention is also thereby described in terms of any individual member or subgroup of members of the Markush group or other group.

The following are provided for exemplification purposes only and are not intended to limit the scope of the invention described in broad terms above.

### EXAMPLES

### Materials and Methods for Examples 1-8

A375, M481, M405, and M498 cells were treated for three days in a 96 well plate while suspended in polymerized 3D collagen in order to mimic a 3D microenvironment. Collagen was used at a final concentration of 2 mg/mL from a 3.2 mg/mL stock concentration. To prepare 1 mL of 2 mg/mL collagen 100 μL 10×PBS, 1M NaOH, 25 μL water and 64 μL of 3.2 mg/mL collagen were mixed. The plate was pre-warmed at 37° C. in the incubator and 10×PBS, 1M NaOH and sterile water were pre-warmed in a 37° C. waterbath. Collagen was brought to room temperature. Cells were trypsinized, harvested and counted. Cells were then resuspended in 2 mg/mL collagen and added to the multi-well plate at 30,000 cells/well. Collagen was allowed to polymerize at 37° C. Following collagen polymerization, media was added to the wells and incubated overnight, with drug added the following day.

SU11652 was added to cells at 1 nM, 10 nM, 100 nM, 1 μM, 2.5 μM, 5 μM and 10 μM along with a DMSO control. Cells were treated with the drug for 3 days, followed by viability assays.

To perform viability assays cells were treated with 2 μM Caspase 3/7, 4 μM ethidium homodimer, and the nucleus was stained with Hoechst at 10 μg/mL. Cells were incubated with fluorescent reagents for 30 minutes before imaging.

Imaging was performed using a Nikon Ti epifluorescence microscope equipped with an automated z-stepper. 3D imaging was performed for a total of 500 μm with a Z step of 2.5 μm. The data was then quantified by counting pixels in multiple z-stacks of images using custom software, FoRTE, (available at github.com/Cobanoglu-Lab/FoRTE). Absolute pixel counts from the three channels was reported and transformed to relative pixel count. To determine relative signal (i.e., pixel count), pixels from the three channels were summed to 100% and percentages determined by calculating the ratio of positive pixels for each channel to the sum of pixels from all the channels.

### Example 1

This example describes the effect of SU11652 on BRAF mutant and BRAF-WT melanoma cells.

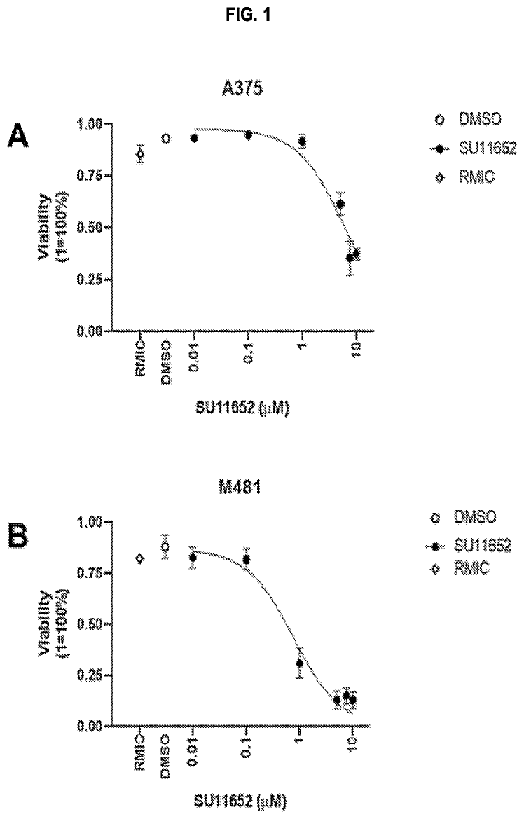

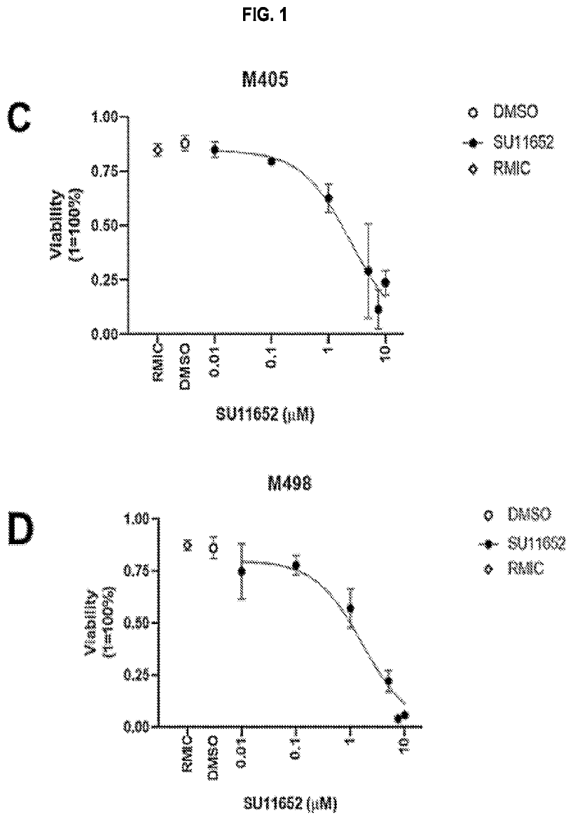

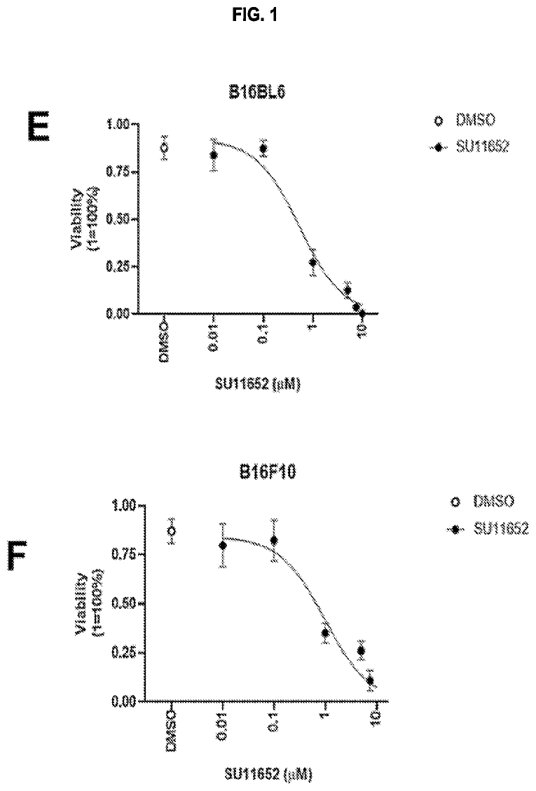

Dose response curves of SU11652 were produced on two BRAF V600E mutant models (A375 cells and patient-derived xenograft M481 cells; FIGS. 1A-B) and on BRAF wild-type, patient-derived xenograft (PDX) cell cultures (M405 and M498; FIG. 1C-D). Cells were incubated with SU11652 at the indicated concentrations, or with DMSO as a negative control, or with RAF/MEK inhibitor combination (RMIC) consisting of 1 μM dabrafenib and 100 nM trametinib as a positive control followed by viability assays. The experiments were conducted in collagen matrix to create 3D culture conditions which are more physiologically relevant than 2D glass or plastic based culture. SU11652 was broadly cytotoxic to melanoma cells and particularly effective on BRAF-WT melanoma cells for which no targeted therapies are available.

These results show that SU11652 effectively killed both BRAF mutant and BRAF-WT melanoma cells.

### Example 2

This example describes the effect of SU11652 on non-cancerous stromal cells.

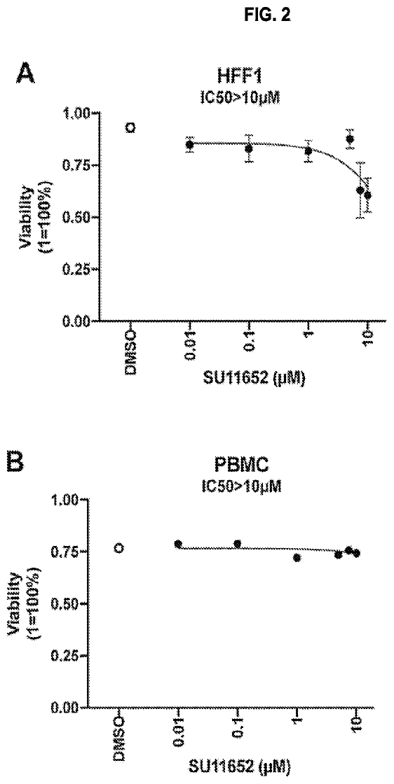

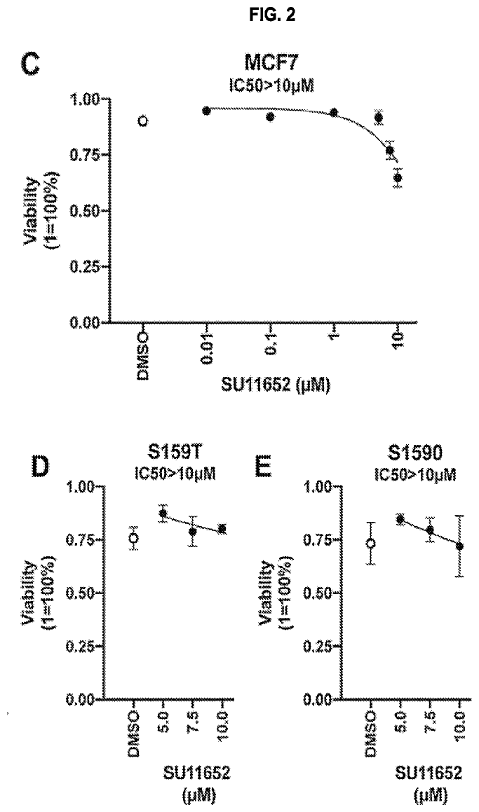

Human foreskin fibroblasts (HFF1) were chosen as representative non-cancerous stromal cells (FIG. 2A) in the human skin. Cells were incubated with SU11652 at the indicated concentrations or with DMSO as a negative control, followed by viability assays. Minimal staining with the ethidium dimer and apoptosis markers was seen for all cells tested, consistent with a minimal effect of SU11652 on cell viability.

Human peripheral blood mononuclear cells (PBMC) were used to assess the impact of SU11652 on the viability of a collection of normal, healthy immune cells of the human body. Cells were isolated from the buffy coats obtained from healthy adult human blood. After culture in ultra-low adhesion dishes, the cells were treated with different doses of SU11652 for 72 hours. The results show that SU11652 did not reduce the viability of PBMCs at the tested doses.

These results show that SU11652 was not cytotoxic to normal or non-cancerous cells, or normal human immune cells, consistent with specificity for tumor cells. These results further show that cytotoxicity of SU11652 was specific to melanoma cells.

### Example 3

This example describes functional differences between SU11652 and Sunitinib.

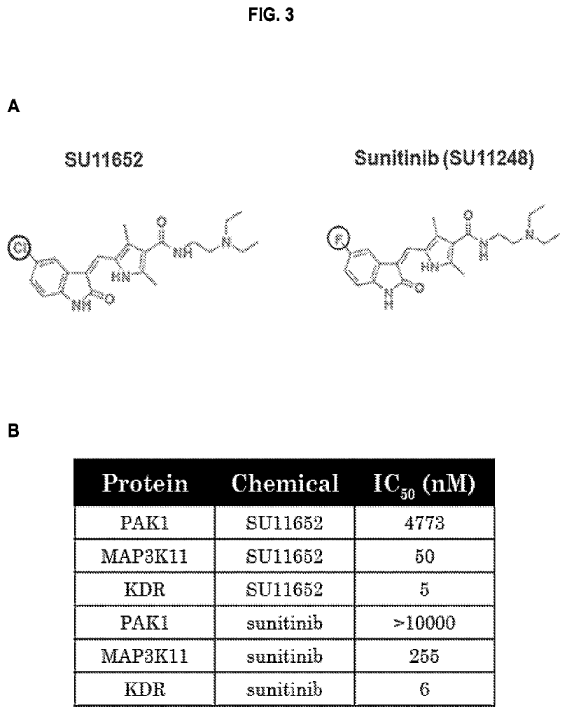

The chemical structures of SU11652 and Sunitinib (SU11248) are shown in FIG. 3A. The structures differ by one atom (circled). Without being limited by theory, the one-atom difference may contribute to functional differences between the compounds.

The differential effect prediction was validated in biochemical experiments (FIG. 3B); the lower the IC50, the more effective the drug at inhibiting the indicated target protein). The effect of SU11652 and Sunitinib on the activity of the SU11652 target MAP3K11 (also known as MLK3) is shown in FIG. 3B. SU11652 inhibits MAP3K11 activity to a greater extent than Sunitinib inhibits MAP3K11 activity. Thus, although SU11652 and Sunitinib are chemically similar, they are functionally different, as seen by the differential effect on the MAP3K11 target. Without being limited by theory, the differential effect of SU11652 and Sunitinib on the MAP3K11 target may be attributed to the structural difference between SU11652 and Sunitinib.

In addition, computational models were generated in the present studies to assess the mechanism of SU11652 activity. The computational models predicted that SU11652 achieves its therapeutic activity through p21-activated kinase 1 (PAK1) that plays a role in BRAF-WT melanoma and drives resistance to MAPK inhibitors in BRAF-mutant melanoma.

The effect of SU11652 and Sunitinib on the activity of PAK1 is also shown in FIG. 3B. PAK1 is a putative target for both BRAF-WT melanoma and BRAF mutant melanoma. SU11652 targets PAK1 activity while Sunitinib does not. In fact, Sunitinib and SU11562 are functionally different across hundreds of kinases. See, Anastassiadis et al., Nat. Biotechnol. 29, 1039-1045 (2011).

These results show that SU11652 and Sunitinib have different effects on cellular targets and melanoma cells despite being chemically similar. Thus, chemical similarity does not allow for prediction of the effect a compound such as SU11652 may have on killing or inhibiting the growth of cancer cells, such as BRAF-WT melanoma cells and BRAF-mutant melanoma cells.

### Example 4

This example describes the cytotoxic specificity of SU11652.

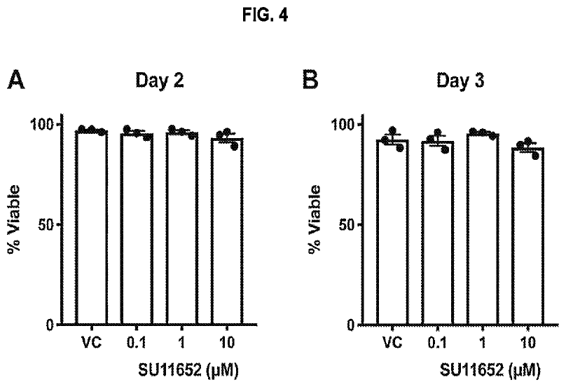

Murine splenocytes were stimulated with anti-CD3, anti-CD28, and murine recombinant IL-2 in addition to the indicated SU11652 doses for 2 and 3 days (FIG. 4). At the indicated timepoints, splenocytes were counted and cell death was quantified by trypan blue staining using an automated cell counter. Error bars represent the standard error of the mean (SEM) for N=3 mice per timepoint. VC=vehicle control. At all SU11652 doses, splenocytes remained viable.

These results show that SU11652 does not kill normal immune cells.

### Example 5

This example describes the effect of SU11652 on cell proliferation and cytokine production.

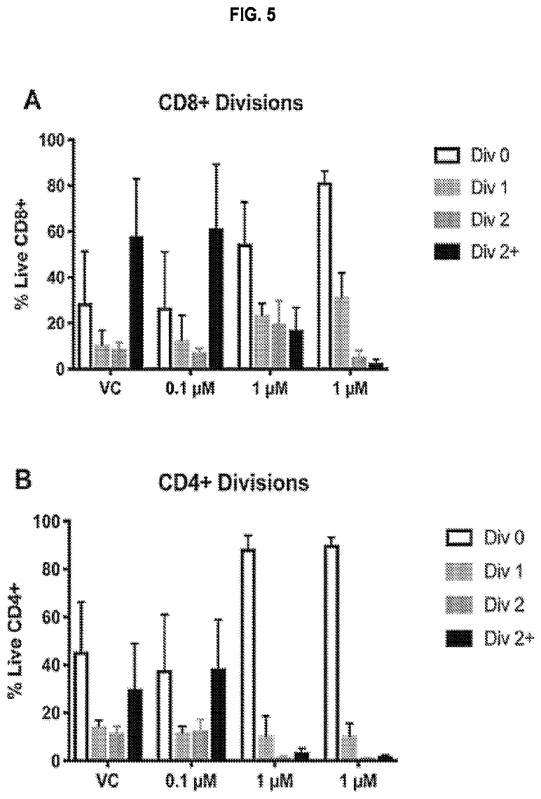

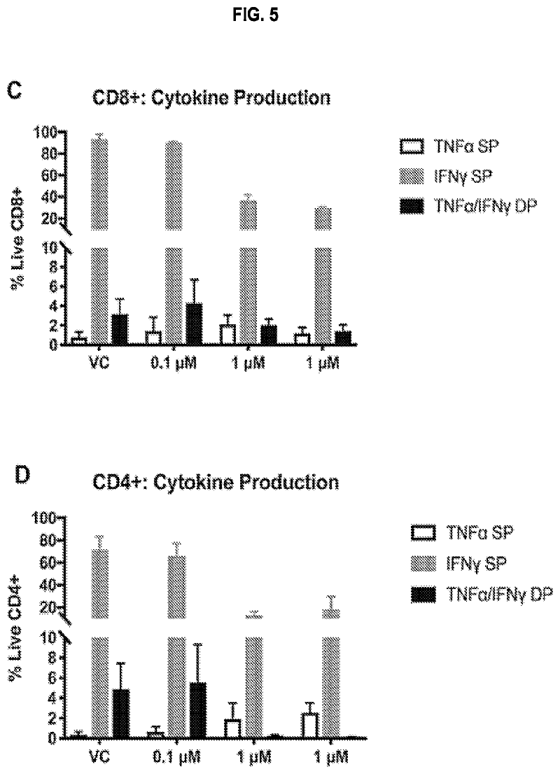

Murine splenocytes were stimulated with anti-CD3, anti-CD28, and murine recombinant IL-2 in addition to the indicated SU11652 doses for 3 days. Prior to stimulation, cells were stained with CFSE to track cell division. Increased concentrations of SU11652 did not impair cell division of both CD8⁺ and CD4⁺ T lymphocytes (FIG. 5, upper; for each graph and each condition, bars show from left to right: first bar Div 0 meaning undivided, second bar Div 1 meaning cells undergone one round of cell division, third bar Div 2, fourth bar Div 2+). Additionally, cells were treated with PMA/lonomycin/Monensin for six hours to detect intracellular cytokines. CD8+ and CD4+ lymphocytes retained their TNFα and IFNγ production abilities (FIG. 5, lower; for each graph and each condition, bars show from left to right: first bar TNFα SP, second bar IFNγ SP, third bar TNFα/IFNγ DP; SP=single positive, DP=double positive). CFSE dilution and intracellular cytokines were detected by flow cytometry. Error bars represent SEM for n=3 mice.

These results show that treatment with SU11652 did not impair splenocyte viability, T lymphocyte proliferation, or cytokine production capacity upon activation. Without being limited by theory, these results provide a basis for successful clinical translation.

### Example 6

This example describes SU11652-mediated reduction of tumor growth in vivo.

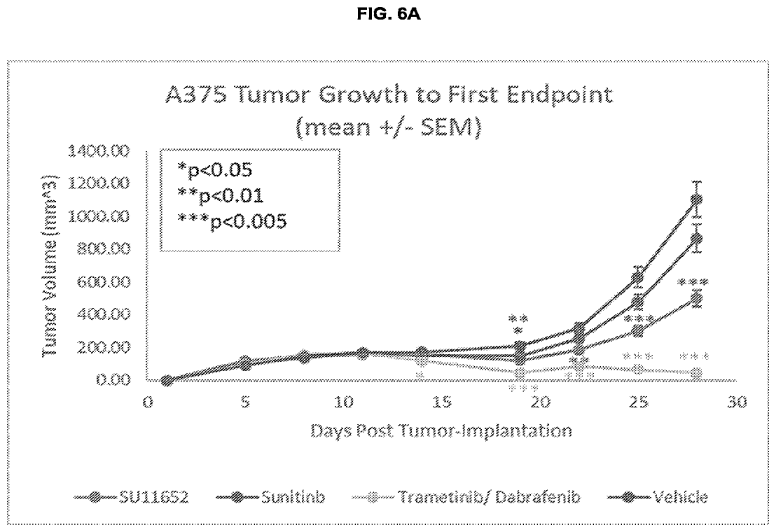

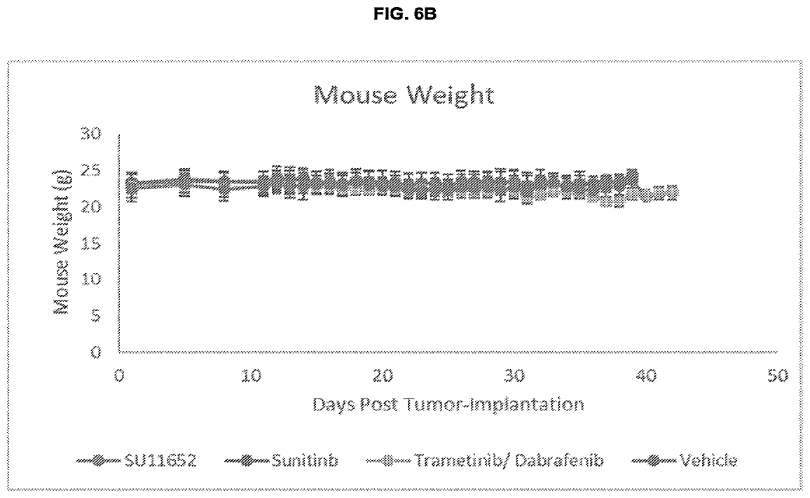

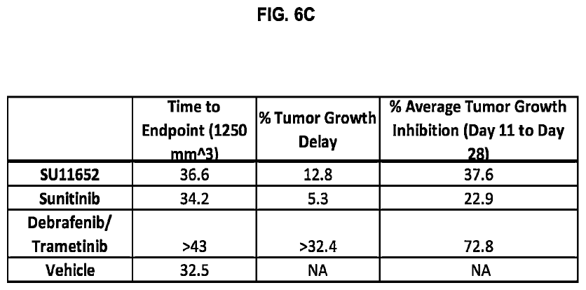

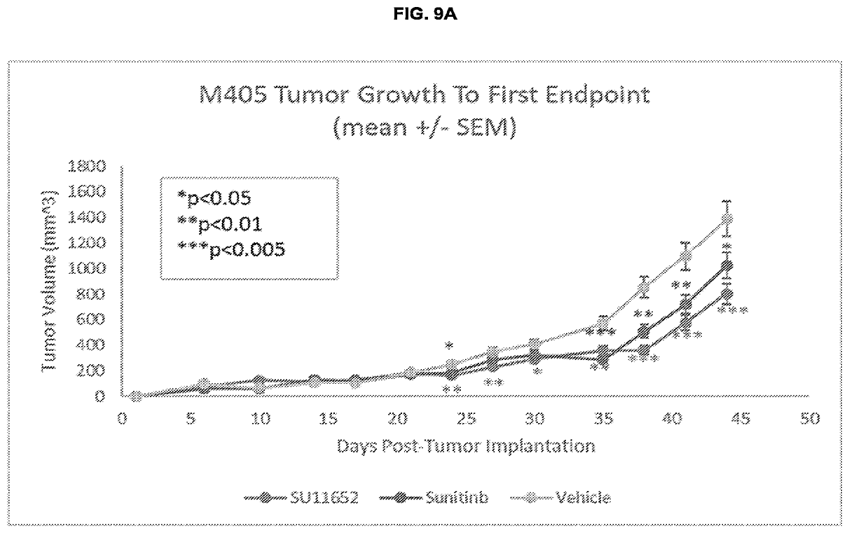

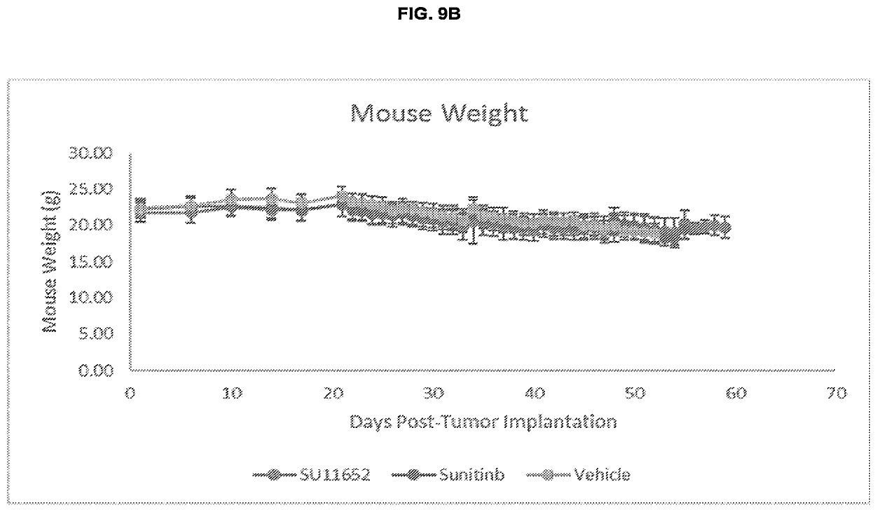

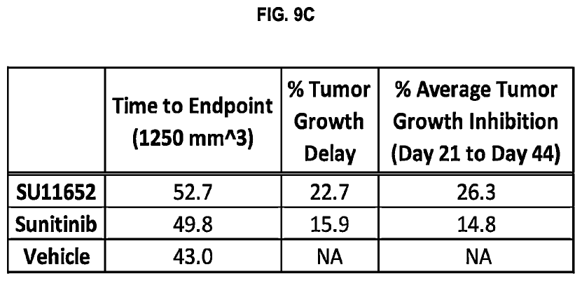

6-8 week old Nod-Scid IL2Rγ KO mice (NSG) were implanted at a single subcutaneous site with 3×10⁶ A375 or M405 melanoma cells (FIGS. 6 and 9, respectively) in matrigel (0.2 ml). When tumors reached approximately 150-200 mm³ (day 11 post tumor cell implantation for A375 cells; day 21 post tumor cell implantation for A405 cells), therapy was initiated. For A375 melanoma cells, treatment was with 20 mg/kg SU11652, 20 mg/kg Sunitinib, 50 mg/kg Debrafenib/0.5 mg/kg Trametinib or vehicle only (0.5% CMC/1.8% NaCl/0.4% Tween80/0.9% Benzyl Alcohol) at 0.2 ml/mouse PO qd. For M405 cells, treatment was with 20 mg/kg SU11652, 20 mg/kg Sunitinib or vehicle only (0.5% CMC/1.8% NaCl/0.4% Tween80/0.9% Benzyl Alcohol) at 0.2 ml/mouse PO qd. Compounds were administered as homogenous slurries after sonication (and heating at 60° C. for debrafenib/trametinib). Mice were weighed daily and tumors were measured with calipers twice weekly. Tumor volume was calculated as (L×W²×pi)/6. When tumor volume reached >1250 mm³ or if mice appeared moribund, they were euthanized and tumors immediately collected and snap frozen. Weight loss did not exceed 20% in any animal and was comparable overall between groups. All mice not previously euthanized were sacrificed on day 43 (A375 cells) or day 59 (M405 cells).

Tumor growth was evaluated as described in Evans (Evans et al., Cancer Immunol Res. 2015 June; 3(6):689-701). Time to endpoint (TTE) was determined by extrapolating the day on which tumors reached 1250 mm³ from log-transformed tumor growth data sets. The data set for each mouse included the first observation that exceeded the endpoint volume and the value that immediately preceded it. The median TTE was calculated for the group and tumor growth delay (TGD) defined as the increase in the median TTE in a treatment group compared to the control group, using the formula: % TGD=((T−C)/C)×10. Percent tumor growth inhibition (TGI) was defined as the difference between the median tumor volume (MTV) of a test group and control group, using the formula: % TGI=((MTVcontrol−MTVtreated/MTVcontrol))×100. The average % TGI was calculated for treatment days when all animals were still present. Mean tumor volumes±SEM were plotted and the difference between treated and control evaluated by Student's unpaired t-test (GraphPad QuickCalcs).

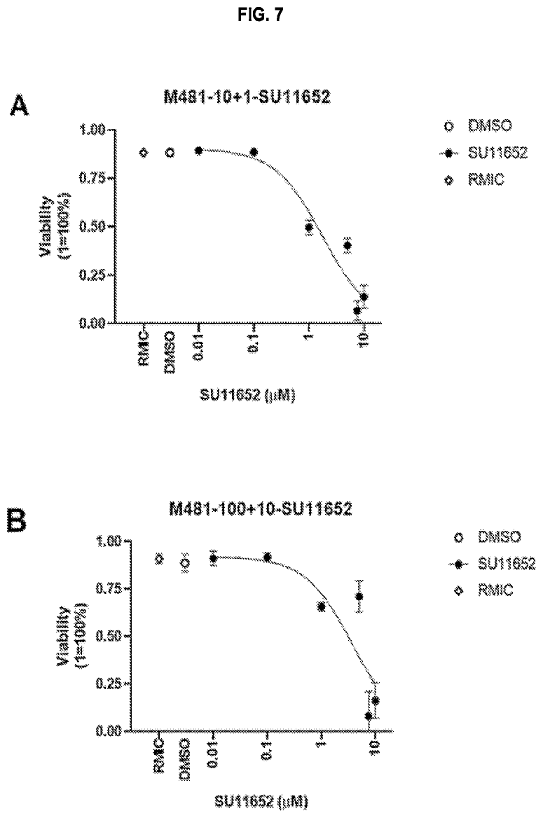

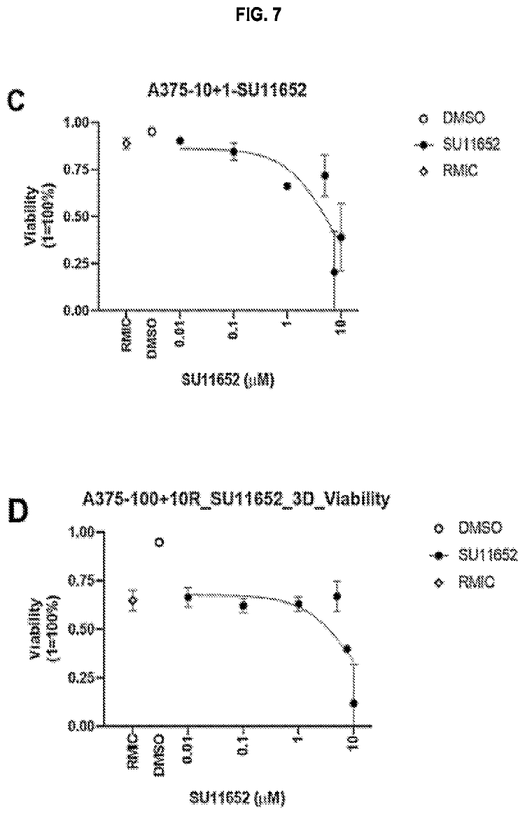

Treatment with SU11652 resulted in a significant reduction over untreated control for both A375 and M405 melanoma tumor cell models (FIGS. 6 and 9, respectively). For tumors with BRAF^{V600E} mutation, treatment with RAF+MEK inhibitors represents effective targeted therapy (FIG. 6). However, in the clinic, this treatment leads to resistance, with a median duration of progression-free survival upon treatment with dabrafenib+trametinib of 11.1 months (95% CI, 9.5 to 12.8). SU11652 worked effectively on BRAF- and MEK-inhibition resistant melanoma cells (FIG. 7) and therefore represents a valuable second line of therapy in the BRAF mutant context.

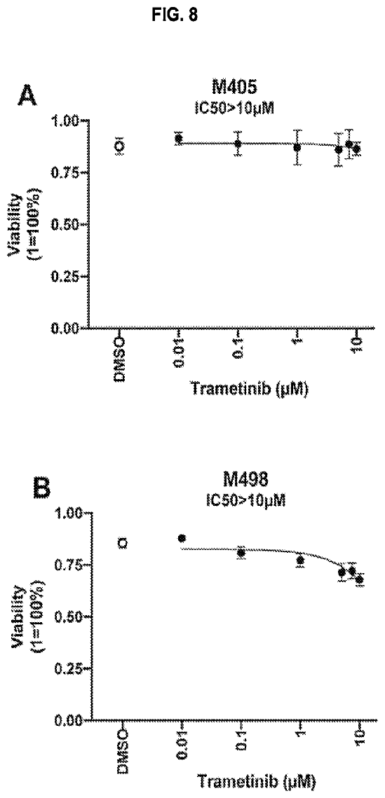

In the BRAF-WT context, MEK inhibition alone showed limited efficacy (FIG. 8), and mutant BRAF inhibitors are also not effective (FIG. 1A-B). Other treatments, such as MEK/AKT inhibition, likewise showed limited efficacy. Therefore, no effective treatment is currently available for a melanoma patient with a BRAF-WT tumor that does not respond to MEK/AKT inhibitors. However, treatment with SU11652 resulted in a significant reduction in tumor volume, as shown in FIG. 9. Furthermore, treatment with the related and clinically utilized kidney cancer drug sunitinib (also known as SU11248) did not result in a similar reduction in tumor volume. Without being limited by theory, based on computational analyses, this difference is likely due to the inhibition of PAK1, a validated target in BRAF-WT melanoma, by SU11652 and not by sunitinib. This differential effect prediction was validated in biochemical experiments (FIG. 3B; the lower the IC50, the more effective the drug at inhibiting the indicated target protein).

These data indicate that SU11652 inhibits PAK1 while sunitinib does not and that SU11652 outperforms sunitinib in vivo in BRAF-WT melanoma, even though a causal relationship between BRAF-WT and melanoma remains to be established. In addition, without being limited by theory, the overall similarity of SU11652 to sunitinib (FIG. 3A) suggests that SU11652 is likely to have favorable pharmacokinetic and pharmacodynamic properties in clinical trials.

In summary, SU11652 is a strong clinical drug candidate for BRAF-WT patients who currently have limited therapeutic options.

Although the present embodiments have been described with reference to specific details of certain embodiments thereof in the above examples, it will be understood that modifications and variations are encompassed within the spirit and scope of the disclosure.

### Example 7

This example describes the effect of SU11652 on immunotherapy suppressive melanoma in vivo.

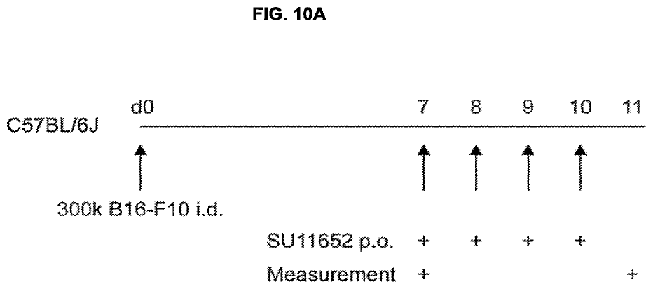

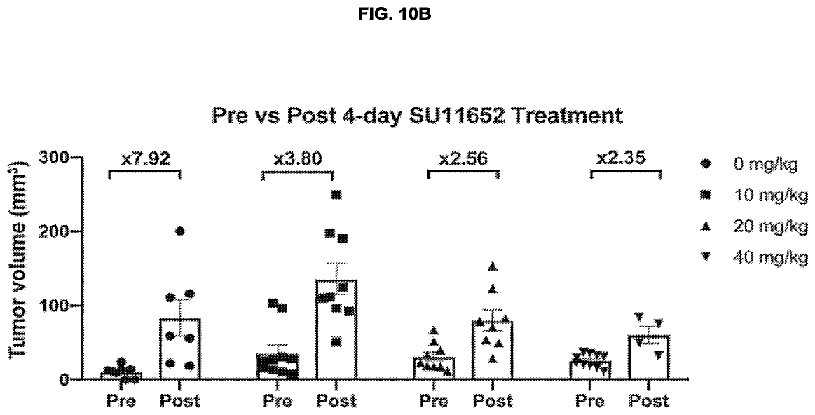

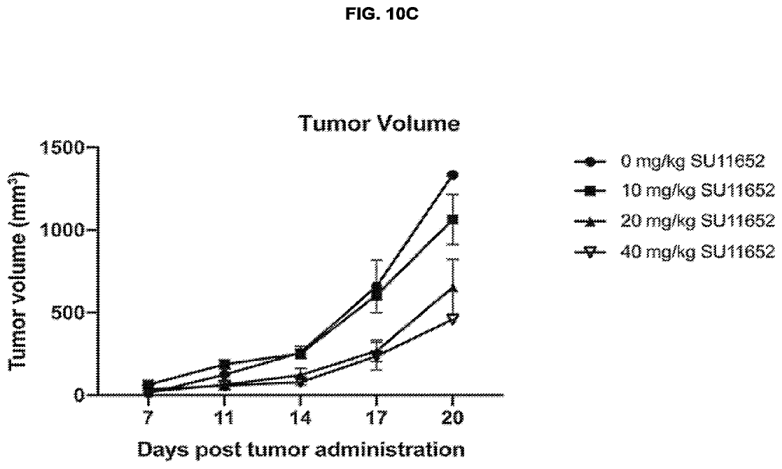

BRAF-WT B16-F10 tumor bearing WT immunocompetent animals were treated with increasing doses of SU11652 and a dose-dependent improvement in tumor control was observed (FIG. 10). Higher doses of SU11652 lead to more tumor control.

These results show that SU11652 has a dose-dependent tumor control effect on immunotherapy suppressive melanoma.

### Example 8

This example describes the repeated and reliable cytotoxic effect of SU11652 melanoma cell lines.

Dose response curves of SU11652 were produced on six different melanoma cells lines. Melanoma cell lines included BRAF V600E mutant models (A375 cells and patient-derived xenograft M481 cells; FIG. 1A-B), BRAF wild-type, patient-derived xenograft (PDX) cell cultures (M405 and M498; FIGS. 1C-D), and B16BL6 and B16F10 cells (both murine melanoma cells) (FIG. 1E-F). Cells were incubated with SU11652 at the indicated concentrations, with DMSO as a negative control, and/or with RAF/MEK inhibitor combination (RMIC) comprising 1 μM dabrafenib and 100 nM trametinib as a positive control followed by viability assays. The experiments were conducted in collagen matrix to create 3D culture conditions which are more physiologically relevant than 2D glass or plastic based culture. SU11652 was broadly cytotoxic to melanoma cells and particularly effective on BRAF-WT melanoma cells for which no targeted therapies are available.

These results show that SU11652 repeatedly, reliably, and effectively killed melanoma cells.

### Example 9

We predicted that the mechanism of action of SU11652 includes blocking the downstream signaling from RAC1. This prediction was grounded on data which shows that SU11652 targets PAK1 and MAP3K11 (FIG. 3B). Furthermore, both RAC1 WT and RAC1 P29S may bind to these two proteins through the CRIB domain and thus may act as downstream effectors of RAC1. Additionally, RAC1 signaling, and especially that of the P29S variant, drives proliferation. However, SU11652 does more than stop proliferation; it kills the melanoma cells in multiple melanoma cell line models (FIG. 1A-F). Therefore, this pathway does not entirely explain that phenotype.

We hypothesized that SU11652 acts by shutting down this downstream signaling from RAC1. This mechanism hypothesis also explains why SU11652 works on RMIC-resistant cell lines (FIG. 7A-D), as PAK signaling drives acquired drug resistance to MAPK inhibitors in BRAF-mutant melanomas.

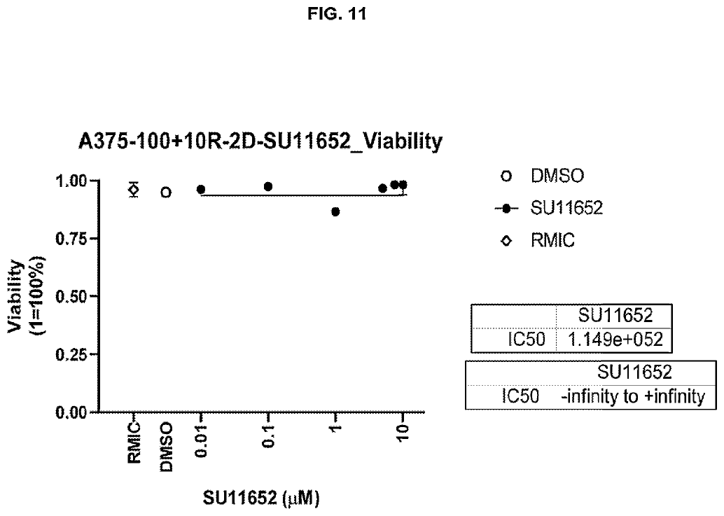

Two new cell lines were used to shed insight into the relationship between RAC1 signaling and SU11652 activity. The first cell line used was IGR1 which is RAC1^{P29S}. This is one of the most common mutations in melanoma and results in an overactive variant of RAC1. A375 is a classical melanoma cell line that is RAC1^{WT} and BRAF^{V600E}, which we had already tested SU11652. The second new cell line was a knock-in RAC1P29S variant of A375 which allows direct comparisons between RAC1^{WT} and RAC1P29s melanoma. It was observed that SU11652 does not show cytotoxic activity on A375 cells in 2D conditions (FIG. 11). Further, the effect of SU11652 under 2D conditions (FIG. 11) did not match that observed in 3D (FIG. 7D) and in vivo. This suggests that the mechanism of SU11652 is mediated through a pathway which is influenced by cell morphology and morphological signaling.

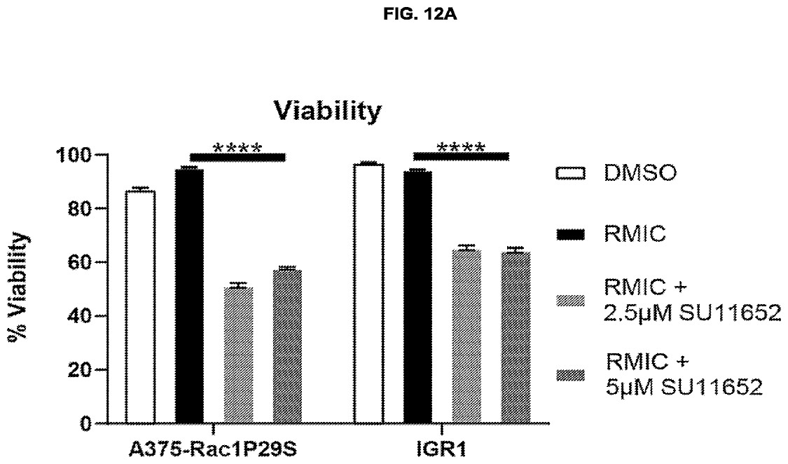

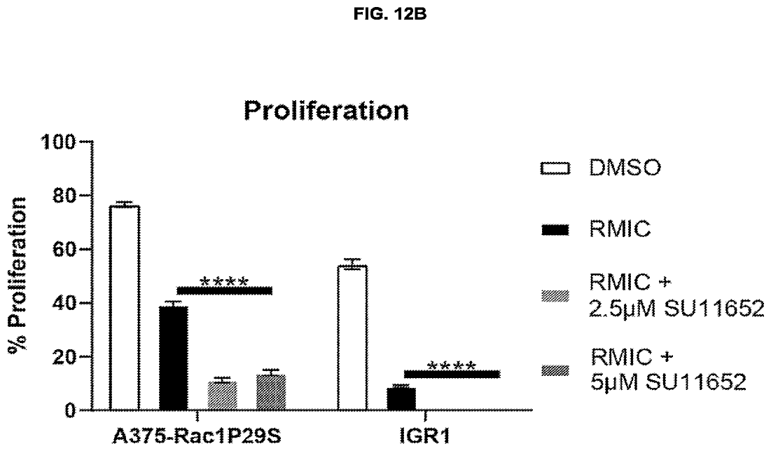

The cytotoxic and cytostatic activity of SU11652 was tested on both RAC1P29S knock-in A375 and the IGR1 (already RAC1^{P29S}) cell lines. RMIC alone did not have any cytotoxic activity, but only some cytostatic activity on these cell lines with hyperactivated RAC1 (FIG. 12A-B). However, the addition of SU11652 caused both cytotoxic and cytostatic effect in both cell lines (FIG. 12A-B).

This data supports the hypothesis that a partial mechanism of action of SU11652 may be to suppress the downstream effectors of RAC1, namely PAK1 and MAP3K11. As mentioned, SU11652 does more than stop proliferation; it kills the melanoma cells in multiple melanoma cell line models (FIG. 1A-F). Therefore, this pathway does not entirely explain that phenotype.

What is claimed is:

1. A method of treating melanoma comprising administering to a subject having melanoma a therapeutically effective amount of SU11652 (5-[(Z)-(5-chloro-2-oxo-1,2-dihydro-3H-indol-3-ylidene)methyl]-N-[2-(diethylamino)ethyl]-2,4-dimethyl-1H-pyrrole-3-carboxamide).

2. The method of claim 1, wherein cells of the melanoma comprise wild-type BRAF.

3. The method of claim 1, wherein cells of the melanoma comprise mutant BRAF.

4. The method of claim 1, wherein administering the SU11652 inhibits MAP3K11 activity, PAK1 activity, VEGFR2 activity, or a combination thereof.

5. The method of claim 1, wherein administering the SU11652 is cytotoxic to melanoma cells.

6. The method of claim 5, wherein cytotoxicity of SU11652 is specific for melanoma cells.

7. The method of claim 5, wherein administering the SU11652 is not cytotoxic to non-cancer cells and non-cytotoxic to breast cancer cells.

8. The method of claim 7, wherein the non-cancer cells are fibroblasts, stromal cells, immune cells, or combinations thereof.

9. The method of claim 8, wherein the immune cells are splenocytes.

10. The method of claim 9, wherein the splenocytes comprise T cells.

11. The method of claim 10, wherein T cell proliferation and cytokine production is unaltered in the presence of SU11652.

12. A method of treating a B-Raf inhibitor and MEK inhibitor resistant melanoma comprising administering to a patient in need thereof a therapeutically effective amount of SU11652 (5-[(Z)-(5-chloro-2-oxo-1,2-dihydro-3H-indol-3-ylidene)methyl]-N-[2-(diethylamino)ethyl]-2,4-dimethyl-1H-pyrrole-3-carboxamide).

13. The method of claim 12, wherein the B-Raf inhibitor is dabrafenib and the MEK inhibitor is trametinib.

14. The method of claim 12, wherein the melanoma comprises a mutant BRAF.

15. A method of inhibiting growth of or killing melanoma cells comprising contacting the melanoma cells with an amount of SU11652 effective to inhibit the growth of or kill the melanoma cells.

16. The method of claim 15, wherein melanoma cells comprise wild-type BRAF.

17. The method of claim 15, wherein melanoma cells comprise mutant BRAF.

18. The method of claim 15, wherein contacting the melanoma cells with the SU11652 inhibits MAP3K11 activity, PAK1 activity, VEGFR2 activity, or combinations thereof.

19. The method of claim 15, wherein contacting the melanoma cells with the SU11652 is cytotoxic to the melanoma cells.

20. The method of claim 19, wherein cytotoxicity of SU11652 is specific to melanoma cells.
